SEQUENCE LISTING (6 sequences)

SEQ ID NO: 1 - protein, 183 aa, synthetic construct
SPPNFTQHVSEQSKVTDRVSRRLIRTYQLYSRTSGKHVQVLANKKINAMAEDGDAHAKLIVETDTFGSRVRIKGAETGFYICMNRRGKLIGKKNGQGKDCIFTEIVLENNYTALQNVKYEGWYMAFTRKGRPRKGSKTRQHQREVHFMKRLPKGHQIAEHRPFDFINYPFNRRTKRTRYSGER

SEQ ID NO: 2 - protein, 188 aa, synthetic construct
SSPNFTQHVREQSLVTDQLSRRLIRTYQLYSRTSGKHVQVLANKRINAMAEDGDPFAKLIVETDTFGSRVRVRGAETGLYICMNKKGKLIAKSNGKGKDCVFTEIVLENNYTALQNAKYEGWYMAFTRKGRPRKGSKTRQHQREVHFMKRLPRGHHTTEQSLRFEFLNYPPFTRSLRGSQRTWAPEPR

SEQ ID NO: 3 - DNA, 552 bp, synthetic construct
  source 1..552: mol_type = other DNA
tccccgcctaattttacacagcatgtgagtgagcaaagtaaggtgacggaccgggtcagccgtagactaatccggacctaccagctttacagccgaaccagtggcaagcacgtgcaagttctggccaacaagaaaatcaacgccatggccgaagatggtgacgctcatgccaagctcatagtggagacggacacatttgggagtcgagttcgaattaaaggagctgaaacaggcttctacatctgtatgaacaggagggggaaactgattggcaagaaaaacggtcaggggaaagactgcattttcacagagatagtcctggagaacaactatacagctctacagaatgtgaagtacgaaggctggtacatggccttcacgcgcaaaggcagaccccgcaagggctccaaaaccaggcaacaccagcgggaagtccacttcatgaagaggctgcccaagggacaccaaatcgcagagcacagaccctttgatttcatcaactaccctttcaacagacggactaaacgcacccgctactcaggagagcgttga

SEQ ID NO: 4 - DNA, 567 bp, synthetic construct
  source 1..567: mol_type = other DNA
tcctcacctaattttacacagcatgtgagggagcagagcctggtgacggatcagctcagccgccgcctcatccggacctaccaactctacagccgcaccagcgggaagcacgtgcaggtcctggccaacaagcgcatcaacgccatggcagaggacggcgaccccttcgcaaagctcatcgtggagacggacacctttggaagcagagtccgagtccgaggagccgagacgggcctctacatctgcatgaacaagaaggggaagctgatcgccaagagcaacggcaaaggcaaggactgcgtcttcacggagattgtgctggagaacaactacacagcgctgcagaatgccaagtacgagggctggtacatggccttcacccgcaagggccggccccgcaagggctccaagacgcggcagcaccagcgtgaggtccacttcatgaagcggctgccccggggccaccacaccaccgagcagagcctgcgcttcgagttcctcaactacccgcccttcacgcgcagcctgcgcggcagccagaggacttgggccccggagccccgatag

SEQ ID NO: 5 - DNA, 33 bp, synthetic construct
  source 1..33: mol_type = other DNA
catgccatggactccccgcctaattttacacag

SEQ ID NO: 6 - DNA, 30 bp, synthetic construct
  source 1..30: mol_type = other DNA
cgcggatcctcaacgctctcctgagtagcg

(19) United States
(12) Patent Application Publication
(10) Pub. No.: US 2026/0183368 A1
(43) Pub. Date: Jul. 2, 2026

(54) APPLICATION OF FGF8 PROTEIN IN THE PREPARATION OF ANTIBACTERIAL AGENTS

(71) Applicant: Huazhong Agricultural University, Wuhan (CN)

(72) Inventors: Yongan ZHANG, Wuhan (CN); Xujie ZHANG, Wuhan (CN); Yazhen HU, Wuhan (CN)

(21) Appl. No.: 19/339,287

(22) Filed: Sep. 24, 2025

(30) Foreign Application Priority Data
Sep. 29, 2024 (CN) 202411374451.1

(51) Int. Cl. A61K 38/18

(52) U.S. Cl. CPC A61K 38/1825

(57) ABSTRACT

The application of FGF8 protein in the preparation of antibacterial agents is provided. Escherichia coli BL21 (DE3) is used to express grass carp FGF8a and murine FGF8b. After purification through Ni²⁺-TED agarose gel column, recombinant proteins of grass carp FGF8a and murine FGF8b are obtained. Both proteins exhibit significant bactericidal effects against Gram-negative bacteria and Gram-positive bacteria, and can be used to prepare broad-spectrum antibacterial agents or antibacterial drugs, demonstrating high application value.

### CROSS-REFERENCE TO RELATED APPLICATIONS

The subject application claims priority on Chinese Patent Application No. CN202411374451.1, filed on Sep. 29, 2024 in China. The contents and subject matter of the Chinese priority application are incorporated herein by reference.

### REFERENCE TO AN ELECTRONIC SEQUENCE LISTING

The contents of the electronic sequence listing (Name of the File: SequenceListing8029wh.xml; Size: 7,829 Bytes; and Date of Creation: Sep. 24, 2025) is herein incorporated by reference in its entirety.

### TECHNICAL FIELD

The present invention pertains to the technical field of genetic engineering, and specifically relates to the application of fibroblast growth factor 8 (FGF8) protein in the preparation of antibacterial agents. The present invention is the first to disclose and verify that FGF8 protein (including grass carp FGF8a protein and/or murine FGF8b protein) exhibits broad-spectrum antibacterial effects.

### BACKGROUND ART

With the development of aquaculture industry, issues such as antibiotic resistance and drug residues caused by excessive antibiotic use have severely constrained the development of aquaculture industry. Therefore, it is pressing to develop novel antibacterial drugs to replace traditional antibiotics in the aquaculture industry. Antimicrobial peptides or proteins are polypeptides or proteins exhibiting broad-spectrum activity against bacteria, fungi, viruses, and protozoa. Recognized as effective antibiotic alternatives thanks to their unique mechanisms of action and reduced tendency to induce resistance, they demonstrate significant potential for disease prevention and control.

Fibroblast growth factor 8 (FGF8), a member of the fibroblast growth factor (FGF) family, is expressed in multiple tissues during the embryonic period and is critical for the formation of various organs and the nervous system. Additionally, FGF8 is present in peripheral blood leukocytes and participates in the formation of normal blood erythrocyte. Given its role in tissue cell proliferation and differentiation, FGF8 holds clinical potential for tissue wound repair.

The present invention is the first to discover that FGF8 protein (including grass carp FGF8a protein and/or murine FGF8b protein) features broad-spectrum antibacterial activity and is suitable for preparing broad-spectrum antibacterial agents.

### SUMMARY OF THE INVENTION

The objective of the present invention is to provide the application of fibroblast growth factor 8 in the preparation of antibacterial agents.

A further objective of the present invention is to provide the application of fibroblast growth factor 8 in the preparation of a medicament for treating or preventing bacterial infections.

To achieve these objectives, the following technical solutions are adopted:

Application of a fibroblast growth factor 8 (FGF8) protein or an encoding gene thereof in the preparation of an antibacterial agent.

Application of a fibroblast growth factor 8 (FGF8) protein or an encoding gene thereof in the preparation of a medicament for treating or preventing bacterial infections.

Preferably, the fibroblast growth factor 8 is fibroblast growth factor 8 from a mammal and/or an oviparous animal.

Preferably, the mammal is a mouse; the oviparous animal is a fish.

Preferably, the fibroblast growth factor 8 is grass carp FGF8a protein and/or murine FGF8b protein; the grass carp FGF8a protein comprises the sequence as set forth in SEQ ID NO.1; the murine FGF8b protein comprises the sequence as set forth in SEQ ID NO.2.

Preferably, the fibroblast growth factor 8 comprises a protein purification tag.

Preferably, the encoding gene of the grass carp FGF8a protein is set forth in SEQ ID NO.3, and the encoding gene of the murine FGF8b protein is as set forth in SEQ ID NO.4.

Preferably, the bacteria are Gram-negative bacteria and/or Gram-positive bacteria;

Preferably, the Gram-negative bacteria comprise:
Escherichia coli (E. coli), Aeromonas hydrophila (A. hydrophila), Yersinia ruckeri (Y. ruckeri), Pseudomonas fluorescens (P. fluorescens), Edwardsiella ictaluri (E. ictaluri), Vibrio fluvialis (V. fluvialis), and Aeromonas sobria (A. sobria);
the Gram-positive bacteria comprise: Staphylococcus aureus (S. aureus), Streptococcus agalactiae (S. agalactiae), Micrococcus luteus (M. luteus), and Streptococcus dysgalactiae (S. dysgalactiae).

Compared with the existing technology, the present invention has the following beneficial effects:

The present invention is the first to discover that fibroblast growth factor 8 (FGF8) from different animals exhibits broad-spectrum antibacterial effects, demonstrating high efficacy against both Gram-negative and Gram-positive bacteria.

In the present invention, both grass carp FGF8a and murine FGF8b are expressed and purified by using prokaryotic expression. Both FGF8 proteins showed significant bactericidal effects against Gram-negative bacteria and Gram-positive bacteria, indicating broad-spectrum antibacterial activity, and are suitable for preparing broad-spectrum antibacterial agents or antibacterial drugs.

### BRIEF DESCRIPTION OF THE DRAWINGS

FIG. 1 shows purification analysis results of recombinant grass carp FGF8a protein in Embodiment 1 of the present invention, where Lane M: Protein Marker; Lane 1: His-grass carp FGF8a fusion protein.

FIGS. 2A and 2B show antibacterial activity test results of recombinant grass carp FGF8a protein in Embodiment 2 of the present invention, where FIG. 2A shows the antibacterial activity test results of recombinant grass carp FGF8a protein against Gram-negative bacteria; and FIG. 2B shows the antibacterial activity test results of the recombinant grass carp FGF8b protein against Gram-positive bacteria. The vertical axis in both FIGS. 2A and 2B represents bacterial growth (%).

FIG. 3 shows purification analysis results of recombinant murine FGF8b protein in Embodiment 1 of the present invention, where Lane M: Protein Marker; Lane 1: His-murine FGF8b fusion protein.

FIGS. 4A and 4B shows antibacterial activity test results of recombinant murine FGF8b protein in Embodiment 2 of the present invention, where FIG. 4A shows the antibacterial activity test results of recombinant mouse FGF8b protein against Gram-negative bacteria; and FIG. 4B shows the antibacterial activity test results of recombinant mouse FGF8b protein against Gram-positive bacteria. The vertical axis in both FIGS. 4A and 4B represents bacterial growth (%).

FIGS. 5A to 5E show both recombinant grass carp FGF8a protein and recombinant mouse FGF8b protein can bind to various bacteria, where FIG. 5A shows the binding of recombinant grass carp FGF8a protein to Gram-negative bacteria; FIG. 5B shows the binding of recombinant grass carp FGF8a protein to Gram-positive bacteria; FIG. 5C shows the binding of recombinant grass carp FGF8a protein to E. coli and S. aureas is concentration-dependent; FIG. 5D shows the binding of recombinant mouse FGF8b protein to Gram-negative and Gram-positive bacteria; and FIG. 5E shows the binding of recombinant mouse FGF8b protein to E. coli and S. aureas is concentration-dependent. In FIG. 5C, the upper panel for E. coli and the lower panel for S. aureas are the binding of gcFGF8a; and in FIG. 5E, the upper panel for E. coli and the lower panel for S. aureas are the binding of mFGF8b.

FIGS. 6A to 6C show the recombinant grass carp FGF8a protein and the recombinant mouse FGF8b protein show concentration-dependent binding activity towards LPS, PGN, and LTA, where FIG. 6A shows the binding activity of different concentrations of gcFGF8a, mFGF8b, and PBS to LPS; FIG. 6B shows the binding activity of different concentrations of gcFGF8a, mFGF8b, and PBS to PGN; and FIG. 6C shows the binding activity of different concentrations of gcFGF8a, mFGF8b, and PBS to LTA. The vertical axis in FIGS. 6A to 6C represents absorbance.

FIGS. 7A to 7H show the effect of recombinant grass carp FGF8a and recombinant mouse FGF8b proteins on the membrane permeability of Gram-negative and Gram-positive bacteria, where FIG. 7A shows the quantitative analysis of the effect of gcFGF8a on the membrane permeability of different Gram-negative bacteria; FIG. 7B shows the effect of gcFGF8a on the membrane permeability of various Gram-negative bacteria exhibits a concentration dependency; FIG. 7C shows the quantitative analysis of the effect of gcFGF8a on the membrane permeability of different Gram-positive bacteria; FIG. 7D shows the effect of gcFGF8a on the membrane permeability of various Gram-positive bacteria exhibits a concentration dependency; FIG. 7E shows the quantitative analysis of the effect of mFGF8b on the membrane permeability of different Gram-negative bacteria; FIG. 7F shows the effect of mFGF8b on the membrane permeability of various Gram-negative bacteria exhibits a concentration dependency; FIG. 7G shows the quantitative analysis of the effect of mFGF8b on the membrane permeability of different Gram-positive bacteria; and FIG. 7H shows the effect of mFGF8b on the membrane permeability of various Gram-positive bacteria exhibits a concentration dependency. The vertical axis in FIGS. 7B, 7D, 7F, and 7H represents Pl update (%).

FIGS. 8A to 8D show both Gram-negative and Gram-positive bacteria exhibited membrane damage and intracellular content leakage after treatment with grass carp FGF8a and mouse FGF8b, as revealed by transmission electron microscopy and scanning electron microscopy, where FIG. 8A shows the bacterial conditions after treating E. coli and S. aureas with gcFGF8a under transmission electron microscopy at different time intervals; FIG. 8B shows the bacterial conditions after treating E. coli and S. aureas with gcFGF8a under scanning electron microscopy at different time intervals; FIG. 8C shows the bacterial conditions after treating E. coli and S. aureas with gcFGF8b under transmission electron microscopy at different time intervals; and FIG. 8D shows the bacterial conditions after treating E. coli and S. aureas with mFGF8b under scanning electron microscopy at different time intervals. The upper three images in each of FIGS. 8A to 8C are results in E. coli, and the lower three images thereof are results in S. aureaus.

### DETAILED DESCRIPTION OF THE INVENTION

The technical solutions of the present invention will be clearly and completely described below with reference to embodiments. It is apparent that the described embodiments represent only a portion of the embodiments of the present invention, not all embodiments. All other embodiments obtained by ordinary skilled persons in the art based on the embodiments herein without creative efforts shall fall within the protection scope of the present invention.

Recombinant grass carp FGF8a and murine FGF8b proteins with purification tags were obtained for the present invention through prokaryotic expression and their antibacterial effects were verified. Proteins obtained by other methods, such as artificially synthesized grass carp FGF8a and murine FGF8b without purification tags, can also produce antibacterial effects, which will not be elaborated herein due to length limitations.

### Embodiment 1

### Preparation of Recombinant Grass Carp FGF8a and Murine FGF8b Proteins

### (1) Amplification of Grass Carp FGF8a Gene and Synthesis of Murine FGF8b Gene

Specific primers comprising NcoI and BamH I restriction enzyme cutting sites were designed using Primer Premier 5.0 software based on the grass carp FGF8a gene sequence from NCBI and multiple cloning sites of ET-15b vector:

Up (SEQ ID NO: 5):
catg^{a}ccatgg^{b}a^{c} TCCCCGCCTAATTTTACACAG,
Down (SEQ ID NO: 6):
cgc^{d}ggatcc^{e}TCAACGCTCTCCTGAGTAGCG.

Where ^{a} and ^{d} represent protective bases for restriction enzyme cutting sites, ^{b} and ^{e} represent NcoI and BamH I restriction enzyme cutting sites respectively, and c represents an anti-mismatch base.

The target gene FGF8a shown in SEQ ID NO. 3 (which does not comprise signal peptide sequences) was cloned using grass carp cDNA as a template, and the PCR product was recovered using a PCR product recovery kit.

The murine FGF8b gene sequence (as shown in SEQ ID NO. 4) with NcoI and BamH I enzyme cutting sites added at 5′ and 3′ ends respectively was synthesized by Wuhan Tsingke Biotechnology Co., Ltd.

### (2) Construction of BL21-pET-15b-FGF8a and BL21-pET-15b-FGF8b Engineering Strains

The recovered PCR product of grass carp FGF8a gene in Step (1) and pET-15b plasmid were double-digested with NcoI and BamH I, ligated with T4 DNA ligase, and then transformed into DH5α competent cells. Additionally, the synthesized gene sequence of murine FGF8b with NcoI and BamH I enzyme cutting sites was ligated into double-digested pET-15b plasmid and transformed into DH5α competent cells. Positive clones were screened and sequenced. Recombinant plasmids pET-15b-grass carp FGF8a and pET-15b-murine FGF8b were extracted from sequence-verified strains and transformed into BL21 (DE3) expression strains. Positive clones were engineering strains (BL21-pET-15b-FGF8a and BL21-pET-15b-FGF8b) comprising recombinant plasmids pET-15b-grass carp FGF-8a and pET-15b-murine FGF-8b, respectively.

(3) Prokaryotic Expression and Purification of his-Grass Carp FGF8a and his-Murine FGF8b Fusion Proteins

1) Prepare the following buffers: buffer A (pH 8.0): 20 mM Tris, 300 mM NaCl; binding buffer B (pH 8.0): 20 mM Tris, 300 mM NaCl, 8 M urea; wash buffer C (pH 8.0): 20 mM Tris, 300 mM NaCl, 8 M urea, 10 mM imidazole; elution buffer D (pH 8.0): 20 mM Tris, 300 mM NaCl, 8 M urea, 300 mM imidazole; and dialysis buffer E (pH 7.4): 20 mM Tris.

2) Inoculate engineering strains BL21-pET-15b-FGF8a and BL21-pET-15b-FGF8b into an LB medium comprising ampicillin (100 μg/mL), respectively, shake culture at 37° C. until OD₆₀₀ reaches 0.6, add IPTG to a final concentration of 0.75 mM and induce culture at 37° C. for 5 hours.

3) Collect bacterial cells by centrifugation (5,000 g, 10 min), and resuspend them in 50 mL pre-chilled buffer A. Disrupt cells by high-pressure homogenization for 10 min, followed by centrifugation at 12,000 g for 60 min. Collect inclusion body pellets.

4) Dissolve inclusion bodies in 50 mL buffer B. Centrifuge at 12,000 g for 60 min and collect supernatant.

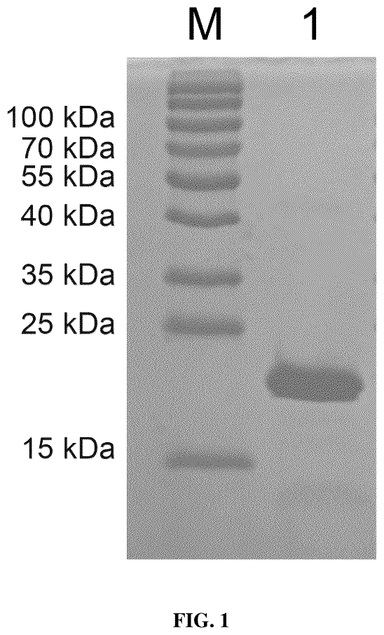

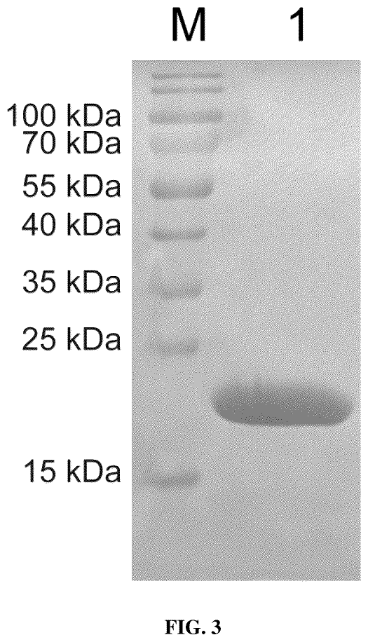

5) Equilibrate Ni2+-TED agarose gel column with 100 mL buffer B. Incubate supernatant with purification matrix at 4° C. overnight. Wash sequentially with 200 mL buffer B and 200 mL buffer C, and elute target proteins with 20 mL buffer D. Then perform dialysis against buffer E with stepwise gradient reduction of urea, imidazole and NaCl concentrations, and obtain His-grass carp FGF8a fusion protein (FIG. 1, comprising the amino acid sequence set forth in SEQ ID NO. 1) and His-murine FGF8b fusion protein (FIG. 3, comprising the amino acid sequence set forth in SEQ ID NO. 2) fused with His tag, respectively.

### Embodiment 2

### In Vitro Antibacterial Activity Assay of Grass Carp FGF8a and Murine FGF8b Proteins

This embodiment validates in vitro broad-spectrum antibacterial activity of grass carp FGF8a and murine FGF8b proteins against 7 Gram-negative bacteria and 4 Gram-positive bacteria that are common in this field, including:

### Gram-Negative Bacteria:

Grass carp FGF8a test group: Escherichia coli (E. coli), Aeromonas hydrophila (A. hydrophila), Yersinia ruckeri (Y. ruckeri), Pseudomonas fluorescens (P. fluorescens), Edwardsiella ictaluri (E. ictaluri), Vibrio fluvialis (V. fluvialis), and Aeromonas sobria (A. sobria);
Murine FGF8b test group: Escherichia coli (E. coli), Aeromonas hydrophila (A. hydrophila), Yersinia ruckeri (Y. ruckeri), and Pseudomonas fluorescens (P. fluorescens);

### Gram-Positive Bacteria:

Grass carp FGF8a test group: Staphylococcus aureus (S. aureus), Streptococcus agalactiae (S. agalactiae), Micrococcus luteus (M. luteus), and Streptococcus dysgalactiae (S. dysgalactiae).
Murine FGF8b test group: Staphylococcus aureus (S. aureus), Streptococcus agalactiae (S. agalactiae), and Micrococcus luteus (M. luteus).

### (1) Bactericidal Activity Assay of Grass Carp FGF8a and Murine FGF8b

1) Streak and incubate the 11 bacterial strains stored at −80° C. onto TSA plates, cultivate them 18 to 24 h at 37° C. for E. coli, Y ruckeri, P. fluorescens, S. aureus, S. agalactiae, M. luteus and S. dysgalactiae, or 28° C. for A. hydrophila, E. ictaluri, V. fluvialis and A. sobria; Inoculate single colonies from the TSA plates into a 20 mL TSB medium. Shake until logarithmic phase (3-5 h), and collect bacterial cells by centrifugation (5,000 g, 10 min);

2) Wash bacteria twice with 20 mM Tris (pH 7.4) (5,000 g, 10 min). Resuspend and adjust the bacterial suspension to 5×10⁷ CFU/mL.

3) Mix bacterial suspensions (2 μL) of grass carp FGF8a protein and murine FGF8b protein test strains respectively with corresponding proteins (48 μL) of varied concentrations or equal volumes of Tris buffer, and incubate at 37° C. or 28° C. for 3 h. Quantify bacterial counts of each group using a combination of spread plate method and colony-forming unit (CFU) counting method.

Equal volume of Tris buffer replaced FGF8a protein as a negative control.

### 4) Calculate the Antibacterial Rate According to the Following Formula:

$$Antibacterial rate ( \% ) = ( Colony count in negative control group - Colony count in experimental group ) / Colony count in negative control group \times 100 \text{} \%$$

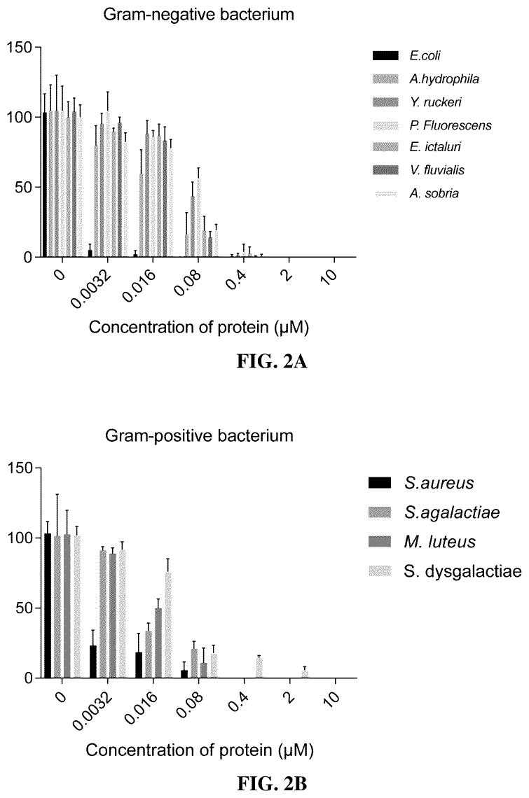

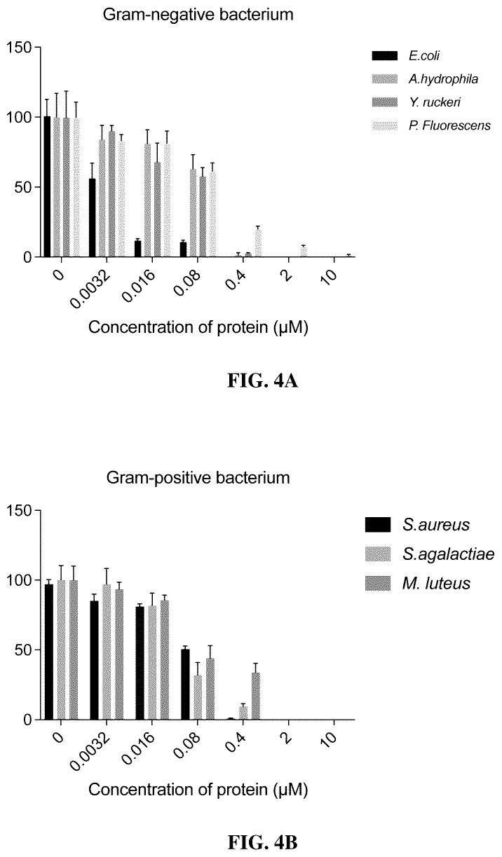

Based on the test results shown in FIGS. 2 and 4, both grass carp FGF8a and murine FGF8b exhibit strong bactericidal activity against Gram-negative and Gram-positive bacteria, demonstrating their suitability for use in the preparation of antibacterial agents.

TABLE 1
Antibacterial Rate of Grass Carp FGF8a against Bacteria

| Final protein | E. | A. | Y. | P. | E. | V. | A. | S. | S. | M. | S. |
|---|---|---|---|---|---|---|---|---|---|---|---|
| concentration (μM) | coli | hydrophila | ruckeri | fluorescens | ictaluri | fluvialis | sobria | aureus | agalactiae | luteus | dysgalactiae |
| 0.0032 | 95% | 20% | 5% | 0% | 10% | 4% | 17% | 77% | 9% | 11% | 12% |
| 0.016 | 98% | 40% | 12% | 14% | 11% | 17% | 22% | 82% | 66% | 50% | 24% |
| 0.08 | 99% | 84% | 56% | 43% | 81% | 86% | 80% | 94% | 79% | 89% | 82% |
| 0.4 | 100% | 99% | 99% | 96% | 97% | 100% | 99% | 100% | 100% | 100% | 85% |
| 2 | 100% | 100% | 100% | 100% | 100% | 100% | 100% | 100% | 100% | 100% | 94% |
| 10 | 100% | 100% | 100% | 100% | 100% | 100% | 100% | 100% | 100% | 100% | 100% |

TABLE 2
Antibacterial Rate of Murine FGF8b against Bacteria

| Final protein | E. | A. | Y. | P. | S. | S. | M. |
|---|---|---|---|---|---|---|---|
| concentration (μM) | coli | hydrophila | ruckeri | fluorescens | aureus | agalactiae | luteus |
| 0.0032 | 58% | 16% | 10% | 16% | 15% | 3% | 6% |
| 0.016 | 91% | 19% | 32% | 18% | 17% | 18% | 14% |
| 0.08 | 92% | 37% | 43% | 36% | 49% | 68% | 56% |
| 0.4 | 100% | 98% | 97% | 80% | 99% | 90% | 66% |
| 2 | 100% | 100% | 100% | 92% | 100% | 100% | 100% |
| 10 | 100% | 100% | 100% | 99% | 100% | 100% | 100% |

### Embodiment 3

### Binding Activity of Grass Carp FGF8a and Murine FGF8b to Bacteria and Pathogen-Associated Molecular Patterns (PAMPs)

This embodiment validates the binding activity of grass carp FGF8a and murine FGF8b proteins to 4 Gram-negative bacteria and 3 Gram-positive bacteria that are common in this field, including:
Gram-negative bacteria: Escherichia coli (E. coli), Aeromonas hydrophila (A. hydrophila), Yersinia ruckeri (Y. ruckeri), and Pseudomonas fluorescens (P. fluorescens);
Gram-positive bacteria: Staphylococcus aureus (S. aureus), Streptococcus agalactiae (S. agalactiae), and Micrococcus luteus (M. luteus).

### (1) Concentration-Dependent Binding Activity Assay of Grass Carp FGF8a and Murine FGF8b to Bacteria

1) Streak and incubate 7 bacterial strains stored at −80° C. onto TSA plates, and cultivate them 18 to 24 h at 37° C. for E. coli, Y. ruckeri, P. fluorescens, S. aureus, S. agalactiae and M. luteus, or 28° C. for A. hydrophila. After 18-24 h, inoculate single colonies into a TSB medium. Shake until logarithmic phase (3-5 h), and collect bacterial cells by centrifugation (5,000 g, 10 min). Wash bacteria three times with 20 mM Tris-HCl (pH 7.4). Resuspend and adjust the bacterial suspension to 1×10⁸ CFU/mL.

2) Mix the 7 bacterial suspensions with equal volumes of grass carp FGF8a or murine FGF8b protein solutions to achieve a final protein concentration of 0.1 μM, and incubate for 1 h at optimal growth temperatures for the corresponding strains.

3) After incubation, centrifuge (5,000 g, 10 min) and wash the bacteria three times (5,000 g, 10 min) with 20 mM Tris (pH 7.4). After discarding the supernatant, resuspend it in 40 μL Tris buffer (20 mM, pH 7.4) and add 10 μL 5×SDS loading buffer. Incubate in a metal bath at 100° C. for 10 min. Then analyze the binding activity of grass carp FGF8a and murine FGF8b to bacteria by Western blot.

4) Using E. coli and S. aureus as representative strains, mix the 2 bacterial suspensions with equal volumes of grass carp FGF8a or murine FGF8b protein solutions to achieve final protein concentrations of 0.02, 0.1, and 0.5 μM. Incubate for 1 h at optimal growth temperatures for the corresponding strains. Analyze concentration-dependent binding of grass carp FGF8a and murine FGF8b to bacteria by Western Blot.

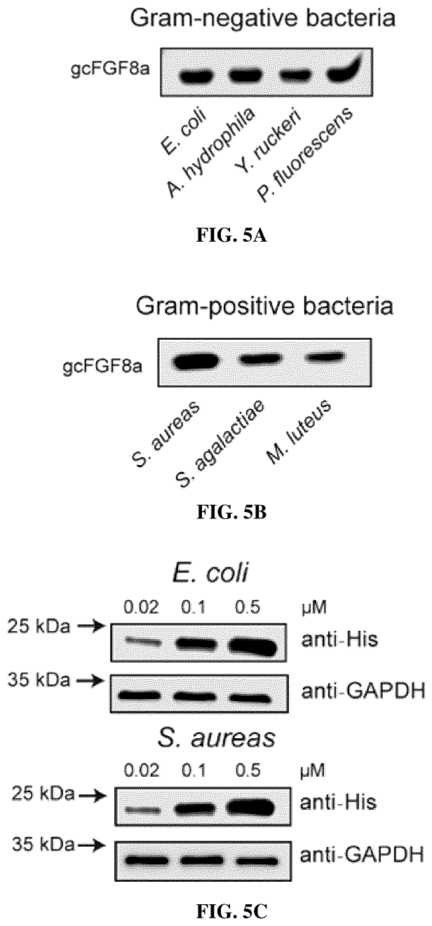

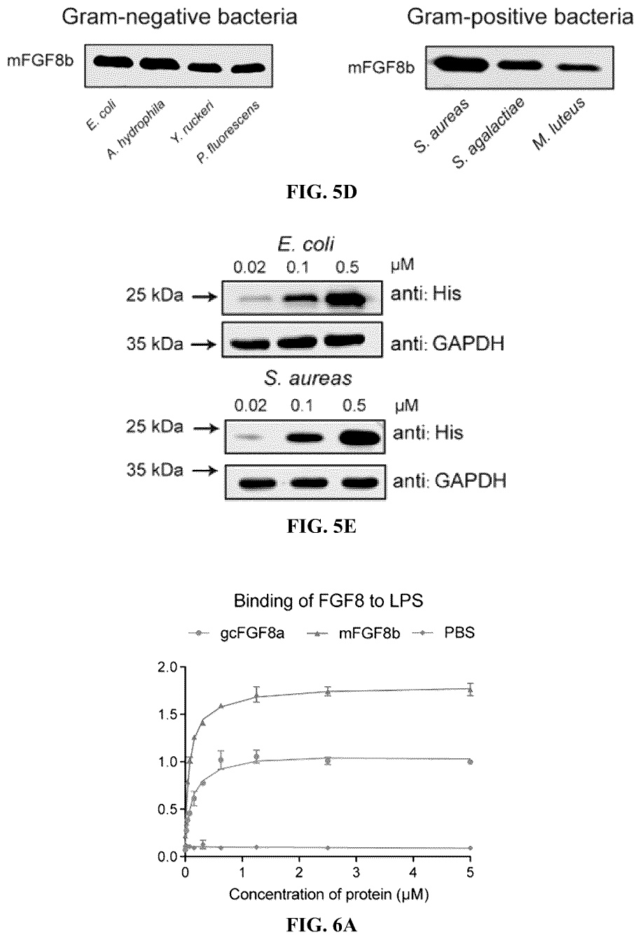

As shown in FIG. 5A to 5E, both grass carp FGF8a and murine FGF8b bind to multiple bacterial species, including four Gram-negative bacteria (E. coli, A. hydrophila, Y. ruckeri and P. fluorescens) and three Gram-positive bacteria (S. aureus, S. agalactiae and M. luteus).

### (2) Binding Activity Assay of Grass Carp FGF8a and Murine FGF8b to PAMPs

1) Dilute PAMPs to a final concentration of 40 ng/L in Na₂CO₃/NaHCO₃ coating buffer. Add 200 μL PAMPs solution to ELISA plates and incubate at 4° C. overnight.

2) Wash three times with PBS (5 min/wash), then add TBST comprising 5% BSA and seal it at 37° C. for 2 h.

3) Wash three times with TBST (5 min/wash). Add to some selected wells 100 μL per well of grass carp FGF8a recombinant protein serially diluted in TBST, and add to another selected wells 100 μL per well of murine FGF8b recombinant protein serially diluted in TBST. Incubate it at 37° C. for 2 h.

4) Wash three times with TBST (5 min/wash). Add 100 μL His-tagged antibody diluted in TBST. Incubate at 37° C. for 1 h.

5) Wash five times with TBST (5 min/wash). Add 100 μL HRP-labeled corresponding secondary antibody diluted in TBST. Incubate it at 37° C. for 40 min.

6) Wash five times with TBST. Add 200 μL TMB substrate per well, and incubate at 37° C. for 30 min. Then add 50 μL stop buffer (2 M H2SO4) per well, and measure absorbance at 405 nm.

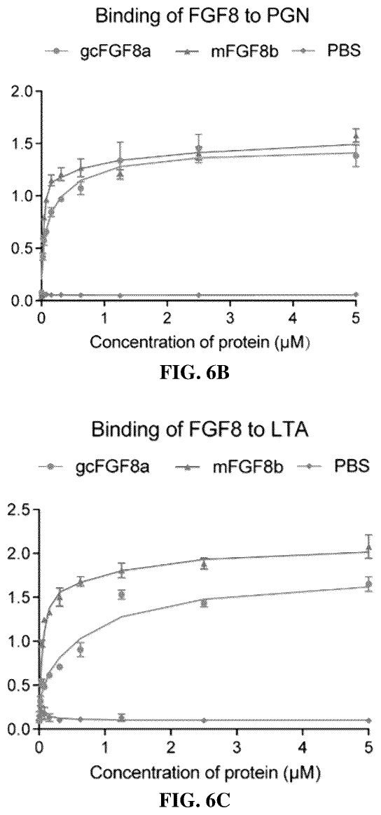

FIGS. 6A to 6C demonstrate concentration-dependent binding of grass carp FGF8a and murine FGF8b to LPS, PGN and LTA.

### Embodiment 4

### Effects of Grass Carp FGF8a and Murine FGF8b on Bacterial Membrane Integrity

(1) Assay of Bacterial Membrane Permeability after Treatment with Grass Carp FGF8a and Murine FGF8b

1) Wash the four Gram-negative bacteria (E. coli, A. hydrophila, Y. ruckeri, and P. fluorescens) and three Gram-positive bacteria (S. aureus, S. agalactiae, and M. luteus) in logarithmic growth phase twice with Tris buffer (20 mM, pH 7.4) and dilute them to a concentration of 5×10⁶ CFU/mL for later use.

2) Incubate the diluted bacterial suspension with grass carp FGF8a and murine FGF8b proteins (final concentrations of 1, 5, and 10 μM) respectively for 1 h at the optimal growth temperature of the corresponding strains.

3) Wash the bacteria twice with 20 mM Tris (pH 7.4), resuspend in an equal volume of Tris (20 mM, pH 7.4), and add propidium iodide (PI) at a final concentration of 10 μg/mL. Incubate in the dark for 30 min.

4) Following washing the bacteria twice with 20 mM Tris (pH 7.4), quantify by flow cytometry the proportion of bacteria exhibiting PI uptake after incubation of grass carp FGF8a and murine FGF8b.

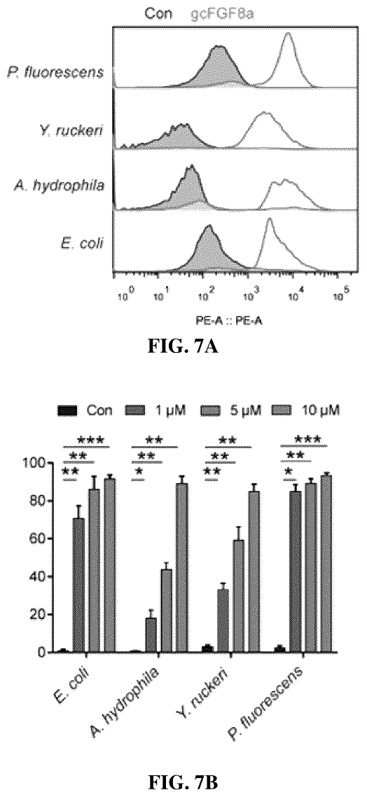

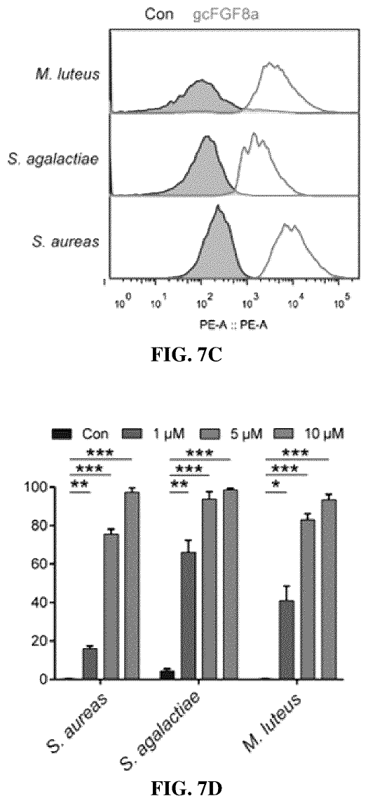

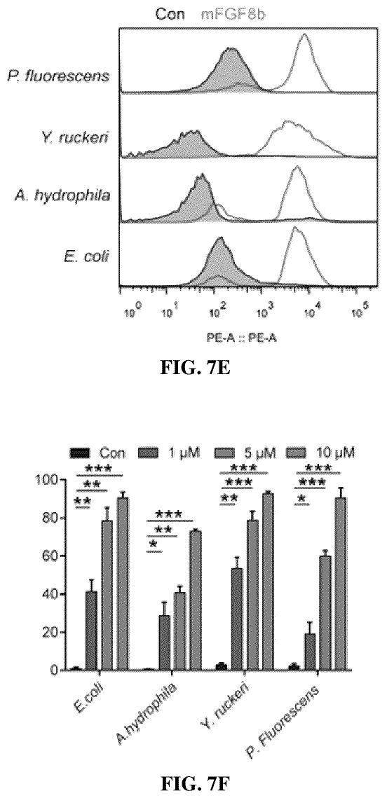

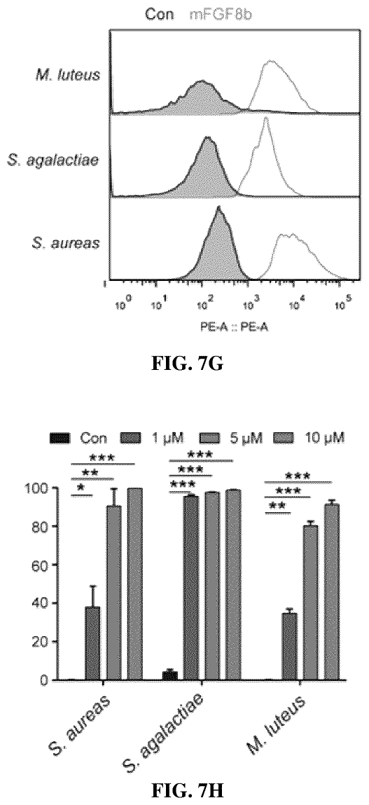

FIGS. 7A to 7H show that both grass carp FGF8a and murine FGF8b can induce changes in bacterial membrane permeability of Gram-negative and Gram-positive bacteria.

(2) Transmission Electron Microscopy (TEM) and Scanning Electron Microscopy (SEM) Imaging of Bacteria after Treatment with Grass Carp FGF8a and Murine FGF8b

Incubate 5×10⁷ CFU of E. coli and S. aureus in logarithmic growth phase with 8 μM grass carp FGF8a protein or murine FGF8b protein at 37° C. for 1 h. For TEM, fix the treated bacteria in 2.5% glutaraldehyde, embed, section, and observe by a TEM (Hitachi H-76500). For SEM, fix the treated bacteria in 2.5% glutaraldehyde, dehydrate through ethanol gradient, dry by vacuum freeze-drying, coat with gold via sputtering, and observe by a SEM (Hitachi S-4800).

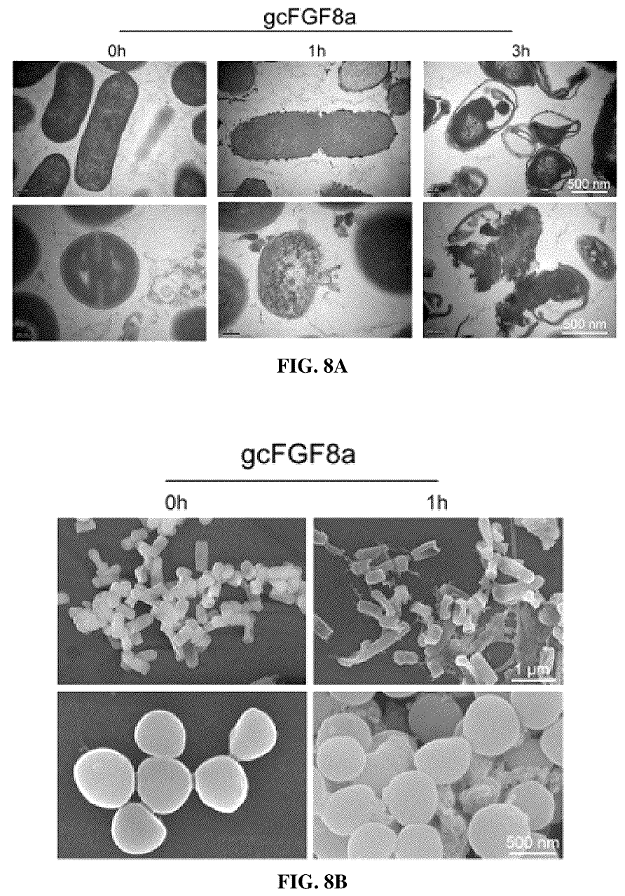

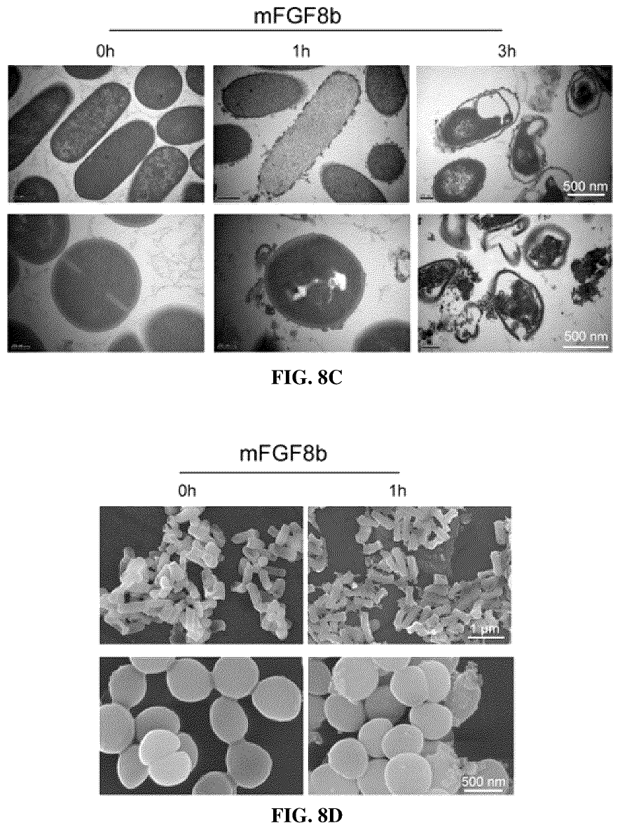

As shown in FIGS. 8A to 8D, TEM and SEM show that both Gram-negative and Gram-positive bacteria exhibit membrane damage and cellular content leakage after treatment with grass carp FGF8a and murine FGF8b, indicating that grass carp FGF8a and murine FGF8b kill Gram-negative and Gram-positive bacteria in a membrane-dependent manner.

While preferred embodiments are described herein, the protection scope of the present invention is not limited thereto. Any modification or substitution readily conceivable by those skilled in the art within the technical scope disclosed herein shall fall within the protection scope of the present invention.

SEQUENCE LISTING

SEQ ID NO: 1
SPPNFTQHVSEQSKVTDRVSRRLIRTYQLYSRTSGKHVQVLANKKINAMAEDGD
AHAKLIVETDTFGSRVRIKGAETGFYICMNRRGKLIGKKNGQGKDCIFTEIVLENNYTAL
QNVKYEGWYMAFTRKGRPRKGSKTRQHQREVHFMKRLPKGHQIAEHRPFDFINYPFNR
RTKRTRYSGER
SEQ ID NO: 2
SSPNFTQHVREQSLVTDQLSRRLIRTYQLYSRTSGKHVQVLANKRINAMAEDGDP
FAKLIVETDTFGSRVRVRGAETGLYICMNKKGKLIAKSNGKGKDCVFTEIVLENNYTALQ
NAKYEGWYMAFTRKGRPRKGSKTRQHQREVHFMKRLPRGHHTTEQSLRFEFLNYPPFT
RSLRGSQRTWAPEPR
SEQ ID NO: 3
TCCCCGCCTA ATTTTACACA GCATGTGAGT GAGCAAAGTA AGGTGACGGA CCGGGTCAGC 60
CGTAGACTAA TCCGGACCTA CCAGCTTTAC AGCCGAACCA GTGGCAAGCA CGTGCAAGTT 120
CTGGCCAACA AGAAAATCAA CGCCATGGCC GAAGATGGTG ACGCTCATGC CAAGCTCATA 180
GTGGAGACGG ACACATTTGG GAGTCGAGTT CGAATTAAAG GAGCTGAAAC AGGCTTCTAC 240
ATCTGTATGA ACAGGAGGGG GAAACTGATT GGCAAGAAAA ACGGTCAGGG GAAAGACTGC 300
ATTTTCACAG AGATAGTCCT GGAGAACAAC TATACAGCTC TACAGAATGT GAAGTACGAA 360
GGCTGGTACA TGGCCTTCAC GCGCAAAGGC AGACCCCGCA AGGGCTCCAA AACCAGGCAA 420
CACCAGCGGG AAGTCCACTT CATGAAGAGG CTGCCCAAGG GACACCAAAT CGCAGAGCAC 480
AGACCCTTTG ATTTCATCAA CTACCCTTTC AACAGACGGA CTAAACGCAC CCGCTACTCA 540
GGAGAGCGTT GA 552
SEQ ID NO: 4
TCCTCACCTA ATTTTACACA GCATGTGAGG GAGCAGAGCC TGGTGACGGA TCAGCTCAGC 60
CGCCGCCTCA TCCGGACCTA CCAACTCTAC AGCCGCACCA GCGGGAAGCA CGTGCAGGTC 120
CTGGCCAACA AGCGCATCAA CGCCATGGCA GAGGACGGCG ACCCCTTCGC AAAGCTCATC 180
GTGGAGACGG ACACCTTTGG AAGCAGAGTC CGAGTCCGAG GAGCCGAGAC GGGCCTCTAC 240
ATCTGCATGA ACAAGAAGGG GAAGCTGATC GCCAAGAGCA ACGGCAAAGG CAAGGACTGC 300
GTCTTCACGG AGATTGTGCT GGAGAACAAC TACACAGCGC TGCAGAATGC CAAGTACGAG 360
GGCTGGTACA TGGCCTTCAC CCGCAAGGGC CGGCCCCGCA AGGGCTCCAA GACGCGGCAG 420
CACCAGCGTG AGGTCCACTT CATGAAGCGG CTGCCCCGGG GCCACCACAC CACCGAGCAG 480
AGCCTGCGCT TCGAGTTCCT CAACTACCCG CCCTTCACGC GCAGCCTGCG CGGCAGCCAG 540
AGGACTTGGG CCCCGGAGCC CCGATAG 567

We claim:

1. A method of preparing an antibacterial agent, comprising
using a fibroblast growth factor 8 protein or an encoding gene thereof, and
preparing an antibacterial agent comprising the fibroblast growth factor 8 protein or the encoding gene thereof.

2. A method for preparing a medicament for treating or preventing bacterial infection, comprising
using the antibacterial agent prepared by the method of claim 1, and
preparing a medicament for treating or preventing bacterial infections comprising the antibacterial agent prepared by the method of claim 1.

3. The method of claim 1, wherein the fibroblast growth factor 8 is fibroblast growth factor 8 from a mammal, an oviparous animal, or both.

4. The method of claim 3, wherein the mammal is a mouse, and the oviparous animal is a fish.

5. The method of claim 4, wherein the fibroblast growth factor 8 is grass carp FGF8a protein, a murine FGF8b protein, or both, the grass carp FGF8a protein comprises the sequence as set forth in SEQ ID NO.1, and the murine FGF8b protein comprises the sequence as set forth in SEQ ID NO.2.

6. The method of claim 1, wherein the fibroblast growth factor 8 comprises a protein purification tag.

7. The method of claim 1, wherein: the bacteria being treated are Gram-negative bacteria, Gram-positive bacteria, or both.

8. The method of claim 7, wherein the Gram-negative bacteria comprise:
Escherichia coli (E. coli), Aeromonas hydrophila (A. hydrophila), Yersinia ruckeri (Y. ruckeri), Pseudomonas fluorescens (P. fluorescens), Edwardsiella ictaluri (E. ictaluri), Vibrio fluvialis (V. fluvialis), and Aeromonas sobria (A. sobria); and
the Gram-positive bacteria comprise: Staphylococcus aureus (S. aureus), Streptococcus agalactiae (S. agalactiae), Micrococcus luteus (M. luteus), and Streptococcus dysgalactiae (S. dysgalactiae).

9. The method of claim 5, wherein the encoding gene of the grass carp FGF8a protein is as set forth in SEQ ID NO.3, and the encoding gene of the murine FGF8b protein is as set forth in SEQ ID NO.4.
